(19) United States
(12) Patent Application Publication
(10) Pub. No.: US 2016/0157792 A1
(43) Pub. Date: Jun. 9, 2016

(54) FIXED/MOBILE PATIENT HANDLING SYSTEM

(71) Applicant: General Electric Company, Schenectady, NY (US)

(72) Inventors: Sathish Kumar Kambapalli Velayutham, Bangalor (IN); K S Manjunatha, Bangalore (IN); Sathyamurthy Karthikeyan, Bangalore (IN); Shankar Murthy Udupa, Bangalore (IN); Sriram Visvanathan Iyer, Bangalore (IN)

(73) Assignee: General Electric Company, Schenectady, NY (US)

(21) Appl. No.: 14/563,311

(22) Filed: Dec. 8, 2014

(51) Int. Cl. A61B 6/04; A61G 7/018; A61G 7/012

(52) U.S. Cl. CPC A61B 6/0407; A61G 7/012; A61G 7/018

(57) ABSTRACT

A system and method for adjusting the height of a portion of a patient support table to accommodate various types of patients is disclosed. The patient support table includes a center section that is movable relative to first and second sections located on opposite sides of the center section. A lift mechanism is mounted to the support table to raise and lower the center section relative to the stationary first and second sections. When the center section is in its fully raised position, the patient support platform can be moved into and out of the medical scanning device by a platform drive mechanism. The lift mechanism is contained within the patient support table. The platform drive mechanism can be mounted either on the patient support table or on the medical scanning device, depending upon whether the patient support table is stationary or movable.

### BACKGROUND

The present disclosure generally relates to a system and method for adjusting the height of a portion of a patient support table. More specifically, the present disclosure relates to a system and method that adjusts the height of a portion of a patient support table depending upon the type of patient such that the patient support table can cater to the needs of a wide variety of patients and increase the ergonomics of the patient support table.

Medical diagnostic imaging and scanning, systems, such as magnetic resonance imaging (MRI) apparatus, X-Ray machines, positron emission tomography (PET) scanners and computer tomography (CT) scanners are well known in the industry. Such machines are able to carry out medical scanning procedures that are able to diagnose various medical conditions, such as internal injuries, cancerous tumors and the like.

In order to obtain an image of a patient with the different types of CT and MRI scanners, it is necessary that the patient be located exactly at a predetermined position within the scanning device. For such reason, the scanning devices include a patient support table that is movable vertically to properly position the patient relative to the scanner. In addition, a support platform of the patient support table is movable horizontally to move the patient into and out of the scanning device.

To accommodate various patients, the height of a patient support table can be manually adjusted by medical personnel in the testing room. During such adjustment, the entire top surface of the table can be raised or lowered depending upon the patient being loaded onto the table. Currently available adjustable patient support tables raise and lower the entire top surface of the table to initially load the patient onto the table and, after the patient has been loaded, raise the table to the required height for examination.

Although currently available adjustable patient support tables can lower the entire top surface to a lowered height, the lowered height is only a benefit to patients who are mobile. If a patient is on a stretcher, the entire top surface of the patient support table is left at the raised position such that the patient can be transferred onto the patient support table from a stretcher. Further, since the weight of patients in hospitals and clinics is increasing, currently available adjustable patient support tables need to be able to handle larger patients.

### SUMMARY

The present disclosure generally relates to a system and method for adjusting the height of a portion of a patient support table. The center section of the patient support table is movable between a fully lowered height and a fully raised height to cater to the needs of a wide variety of patients and increase the ergonomic capabilities of the patient support table.

The patient support table of the present disclosure is operable to position and support a patient above a floor such that the patient can be supported during a medical scanning procedure carried out by a medical scanning device. The patient support table includes a patient support platform that is movable into and out of the medical scanning device as desired.

The patient support platform of the patient support table includes first and second sections that are each located at a first height above the floor. A center section is positioned between the first and second sections and is movable relative, to the stationary first and second sections. The center section is movable between a raised position in which the center section is at the same height as the first and second sections. In the raised position, the center section, the first section, and the second section are located at the same first height and create the patient support platform that is capable of supporting the patient in a supine position.

The center section can be lowered to a fully lowered position in which the center section is at a second height that is well below the first height. When the center section is in the lowered position, a patient can be seated on the center section.

The patient support table further includes a lift mechanism that is mounted to the support table and is able to raise and lower the center section between the raised and lowered positions. When a patient is initially received on the center section in its fully lowered position, the lift mechanism can be operated to raise the center section from the lowered, second height to the raised, first height. Once the lift mechanism has raised the center section to the raised position, the patient can lay on the fully formed patient support platform.

In a contemplated embodiment of the disclosure, the center section can include a pair of arm boards that are movable with the center section. Each of the pair of arm boards are separately movable between a raised position and a lowered position. When the arm board is in the raised position, the arm board can function as a back rest for a seated patient. When the arm board is in the lowered position, the arm board can function as a thigh support for the seated patient.

In one embodiment of the disclosure, the lift mechanism includes a pair of threaded lead screws that are each coupled to one end of the movable center section. A drive motor that forms a portion of the lift mechanism is operable to rotate the pair of threaded lead screws. Rotation of the threaded lead screws causes the center section to move upward or downward, depending upon the direction of rotation. In this manner, the lift mechanism is able to raise and lower the center section relative to the stationary first and second sections.

In one embodiment of the disclosure, the patient support table is stationary relative to the medical scanning device. In an alternate embodiment, the patient support table is mobile and can be moved away from the medical scanning device. In each embodiment, the lift mechanism used to raise and lower the center section is contained within a base of the patient support table.

The patient support table further includes a platform drive mechanism that is operable to move the patient support platform into and out of the medical scanning device. In the embodiment in which the patient support table is stationary, the platform drive mechanism is mounted to the second section of the patient support table. In the embodiment in which the patient support table is movable, the platform drive mechanism is mounted to a stationary portion of the medical scanning device. In each case, the platform drive mechanism is operable to move the complete patient support platform, including the first section, the center section and the second section, into and out of the medical scanning device. In one embodiment of the disclosure, the platform drive mechanism is a belt drive that engages a pair of belts extending through the medical scanning device. Operation of the belt drive moves the patient support platform into and out of the medical device as desired.

Various other features, objects and advantages of the invention will be made apparent from the following description taken together with the drawings.

### BRIEF DESCRIPTION OF THE DRAWINGS

The drawings illustrate the best mode presently contemplated of carrying out the disclosure. In the drawings:

FIG. 1 is a top end perspective view showing a patient support table positioned in front of a medical scanning device;

FIG. 2 is a side elevation view of the patient support table and medical scanning device;

FIG. 3 is a side perspective view showing a patient on the patient support table with a center section in the lowered position;

FIG. 4 is a top perspective view of the patient support table;

FIG. 5 is a top perspective view showing the center section of the support platform in an intermediate position;

FIG. 6 is a top view showing a patient supported on the patient support table;

FIG. 7 is a view illustrating the belt tracks that move the support platform through the medical scanning device;

FIG. 8 is a view illustrating the belt drive contained within the patient support table;

FIG. 9 is a top end perspective view showing a second embodiment of a patient support table positioned in front of a medical scanning device;

FIG. 10 is a side elevation view of the second embodiment of the patient support table;

FIG. 11 is a perspective view showing the second embodiment of the patient support table with the center section in the fully lowered position;

FIG. 12 illustrates the second embodiment of the patient support table with the center section in an intermediate position;

FIG. 13 is a side view showing the second embodiment of the patient support table with the center section in the fully raised position; and

FIG. 14 is a front perspective view of the medical scanning device including the bridge extension for use with the movable patient support table.

### DETAILED DESCRIPTION

In the present description, certain terms have been used for brevity, clearness and understanding. No unnecessary limitations are to be applied therefrom beyond the requirement of the prior art because such terms are used for descriptive purposes only and are intended to be broadly construed. The different systems and methods described herein may be used alone or in combination with other systems and methods. Various equivalents, alternatives and modifications are possible within the scope of the appended claims. Each limitation in the appended claims is intended to invoke interpretation under 35 U.S.C. §112, sixth paragraph, only if the terms “means for” or “step for” are explicitly recited in the respective limitation.

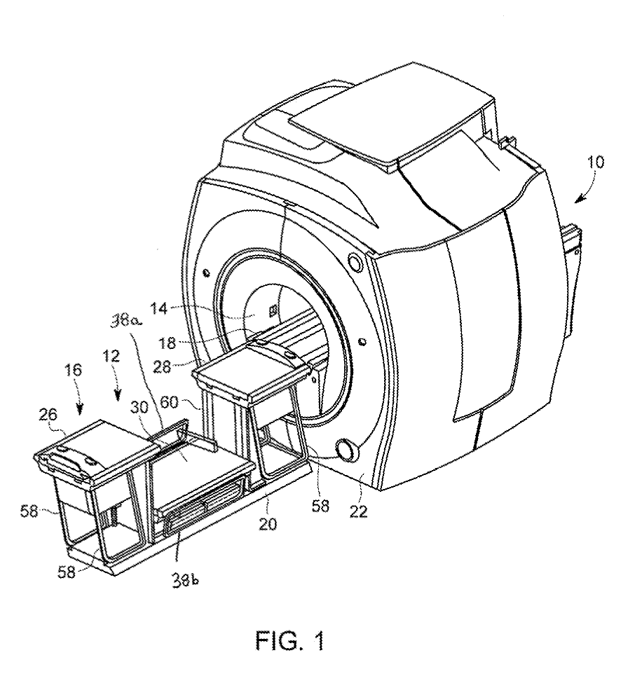

FIG. 1 illustrates a medical scanning device 10 that is shown with an exemplary embodiment of a patient support table 12 constructed in accordance with the present disclosure. As is known to one skilled in the art, the medical scanning device 10 includes an annular gantry 14 containing a field generator that projects a beam of rays toward a detector array on the opposite side of the gantry. The medical scanning device 10 is capable of carrying out a medical scanning procedure on a patient when the patient is supported on a patient support platform 16 of the patient support table 12. The support platform 16 is movable horizontally into and out of the gantry 14 along a guide track 18.

In the embodiment shown in FIG. 1, the patient support table 12 is stationary relative to the medical scanning device 10, although an alternate embodiment shown in later Figures presents the patient support table 12 as a mobile device. The patient support table 12 shown in FIG. 1 is mounted to a stationary base 20. The stationary base 20 is mounted near the front face 22 of the medical scanning device 10.

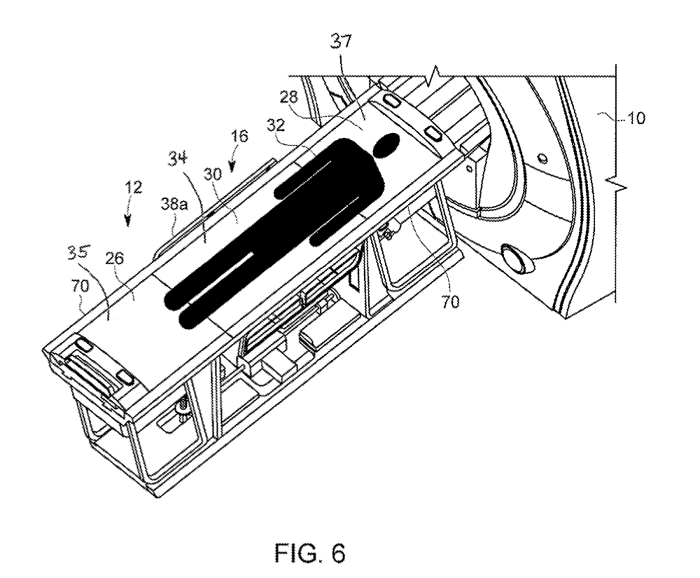

In accordance with the present disclosure, the support platform 16 of the patient support table 12 includes multiple sections. Specifically, the support platform 16 includes a stationary first section 26 and a stationary second section 28 that are positioned on opposite sides of a movable center section 30. The center section 30 is movable from a fully lowered position shown in FIG. 1 to the fully raised position shown in FIG. 6.

As illustrated in FIG. 6, when the center section 30 is in the fully raised position, a generally planar top surface 34 of the center section 30 is coplanar with the top surfaces 35, 37 of both the first and second sections 26 and 28. When in the fully raised position, the first section 26, the second section 28 and the center section 30 can support a patient 32 in the supine position.

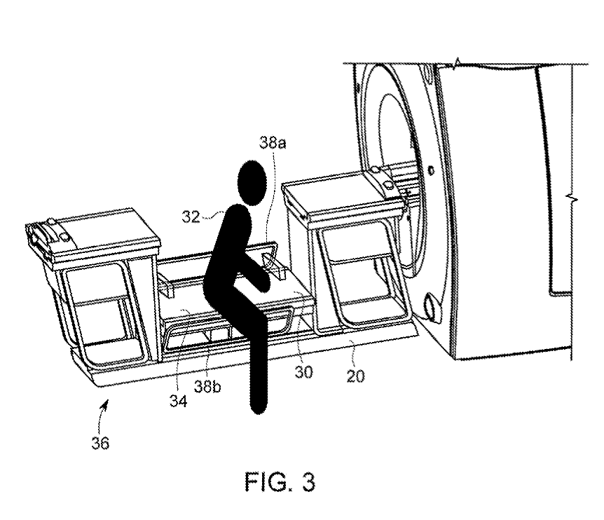

When the center section 30 is in the fully lowered position as shown in FIGS. 1-4, the top surface 34 of the center section 30 is located approximately 350-400 mm above the floor 36 that supports the base 20. When the center section 30 is in this fully lowered position, the patient 32 can more easily sit onto the center section 30 as shown in FIG. 3. In the embodiment shown in FIGS. 1-4, the center section 30 has a length of approximately one meter and is designed to support patients having a weight of up to 750 lbs. These dimensions of the center section and the ability to support a patient up to 750 lbs, allows the patient support. table 12 to accommodate patients of almost all sizes.

The center section 30 includes first and second arm boards 38a and 38b that are positioned on opposite lateral sides of the center section 30. In the condition shown in FIGS. 1-3, the arm board 38b is in a lowered position which allows the patient 32 to be seated on the center section 30, as shown in FIG. 3. The opposite arm board 38a is in a raised position which serves as a back rest for the seated patient 32. When the arm board 38b is in the lowered position, the arm board 38b also serves as a thigh support for the patient when the patient is in the seated position.

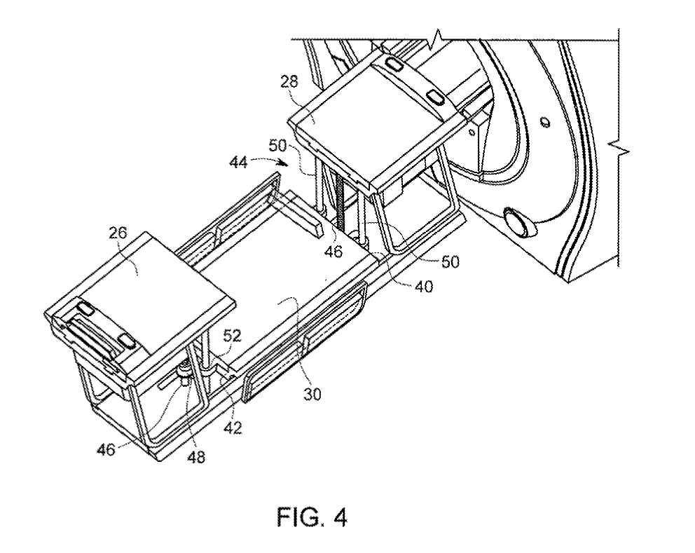

The center section 30 of the patient support table 12 is supported at both its first end 40 and its second end 42 by a lift mechanism 44, which is best shown in FIG. 4. The lift mechanism 44 includes a pair of threaded lead screws 46 that are each received within a threaded collar 48 securely mounted to each of the first and second ends 40, 42 of the center section 30. When the pair of lead screws 46 rotate, the threaded interaction between the lead screw 46 and the collar 48 causes the center section to move either upward or downward relative to the stationary first and second sections 26, 28. In addition to the threaded lead screws 46, each end of the center section 30 moves along a pair of support rods SO located on opposite sides of the lead screw 46. Each of the support rods 50 passes through a support collar 52 to further guide the vertical movement of the center section 30.

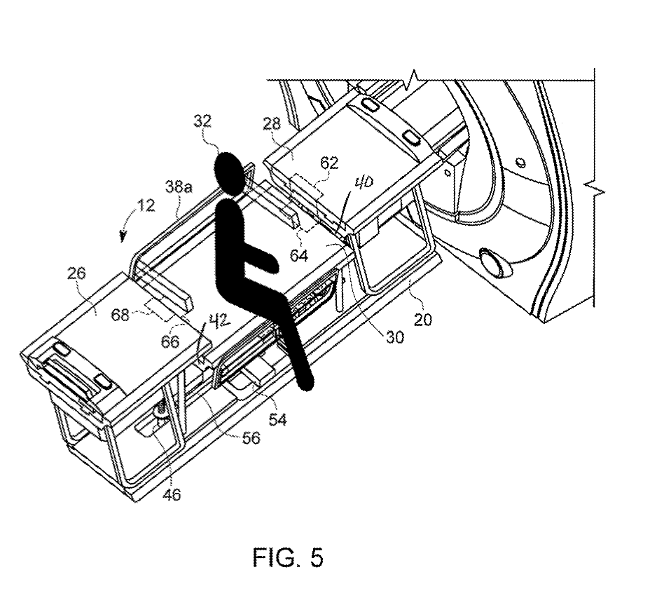

As illustrated in FIG. 5, a drive motor 54 is contained within the base 20. The drive motor 54 operates to rotate a drive shaft 56 which, through a series of gears (not shown), causes rotation of the lead screws 46. The operation of the drive motor 54 can be controlled either remotely or through controls contained on the patient support table 12.

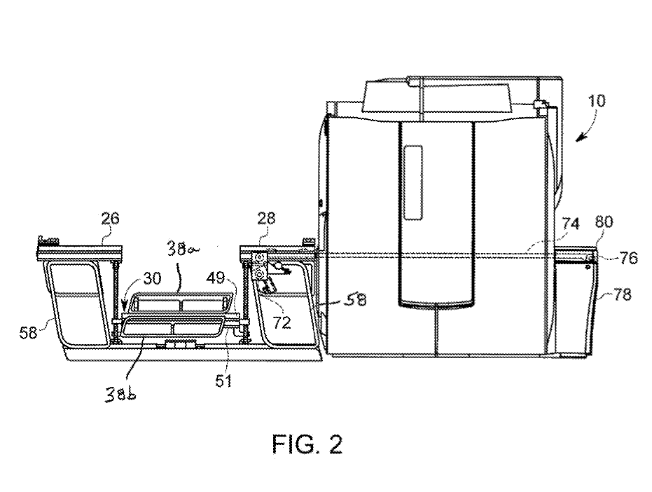

Referring back to FIGS. 1 and 2, the stationary first and second sections 26, 28 of the support platform 16 are each supported at a first height of 965 mm above the floor by a pair of aluminum frames 58. As shown in FIG. 1, a protective shroud 60 conceals the lead screw 46 and support rods 50 at each end of the center section 30.

Referring, back to FIG. 5, once the patient 32 has been seated on the center section 30, the center section 30 can be raised from the lowered position shown in FIG. 4 to the fully raised position shown in FIG. 6. During this movement, the patient 32 is supported on the center section and can lean against the arm board 38a.

As illustrated in FIG. 5, the center section 30 includes a magnetic latch 64 formed near the first end 40 and a second magnetic latch 66 formed near the second end 42. Although the center section 30 is shown as including a pair of magnetic latches 64, 66, it should be understood that mechanical latches could be utilized either in addition to the magnetic latches or in conjunction therewith. The magnetic latch 64 interacts with a similar latch 62 formed on the second section 28 while the magnetic latch 66 engages the latch 68 formed on the first section 26.

Once the center section 30 reaches the fully raised position, the magnetic latch 62 formed in the second section 26 engages a similar latch 64 in the center section 30. In addition to the magnetic latching, a mechanical latch can also be used to join the center section 30 with the second section 28.

The opposite end of the center section includes a similar magnetic latch 66 that engages a latch 68 formed in the first section 26. The latching between the center section 30 and each of the first and second sections 26, 28 occurs only when the center section 30 moves to the fully raised position shown in FIG. 6.

Once the center section 30 is in the fully raised position shown in FIG. 6, the arm board 38 is lowered and the patient is ready to be loaded into the medical scanning device 10. As can be understood in FIG. 6, the support platform 16 of the patient support table 12 is guided by a pair of side rails 70.

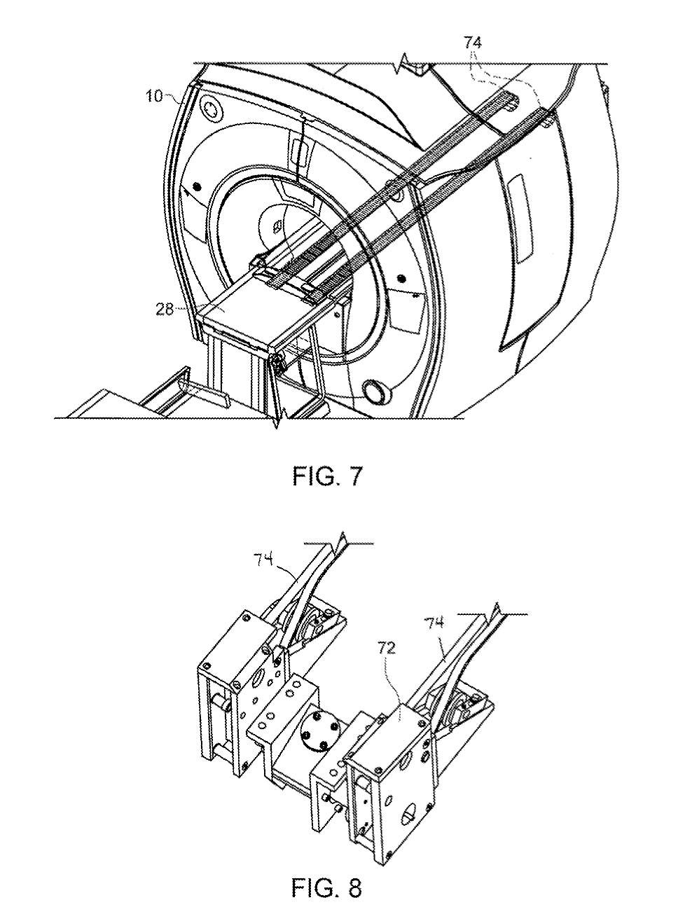

Referring now to FIGS. 7 and 8, the second section 28 of the patient support table includes a platform drive mechanism 72 that works with a pair of belts 74 to move the entire patient support platform into and out of the medical scanning device 10. The dual belt drive mechanism 72 is positioned beneath the second section 28 and moves the entire support platform into and out of the medical scanning device 10. As shown in FIG. 2, each of the belts 74 passes over a pulley 76 contained within the stationary rear pedestal 78. As the dual belt drive 72 operates, the belts cause the entire patient support platform to move into and out of the medical scanning device 10. The support platform can move to the back end 80 of the rear pedestal 78.

As illustrated in FIG. 2, the center section 30 includes a patient support member 49 mounted to the carrier 51. The patient support member 49 can be moved from the carrier 51 once the center section is elevated to be coplanar with the first and second sections 26, 28.

As can be understood in the embodiments shown in FIGS. 1-8, the patient support table 12 is stationary and the lift mechanism 44 is contained within the base while the platform drive mechanism 72 used to move the patient support platform into and out of the medical scanning device 10 is mounted beneath the second section 28 located closest to the medical scanning device 10.

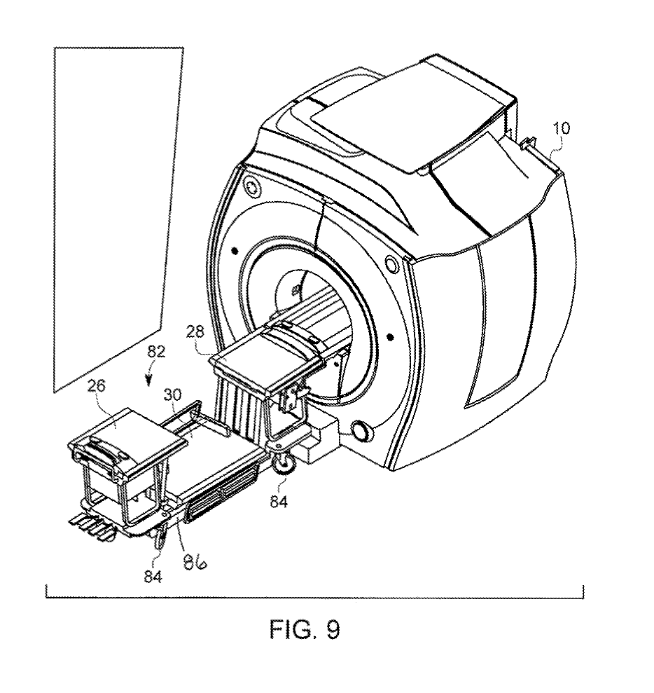

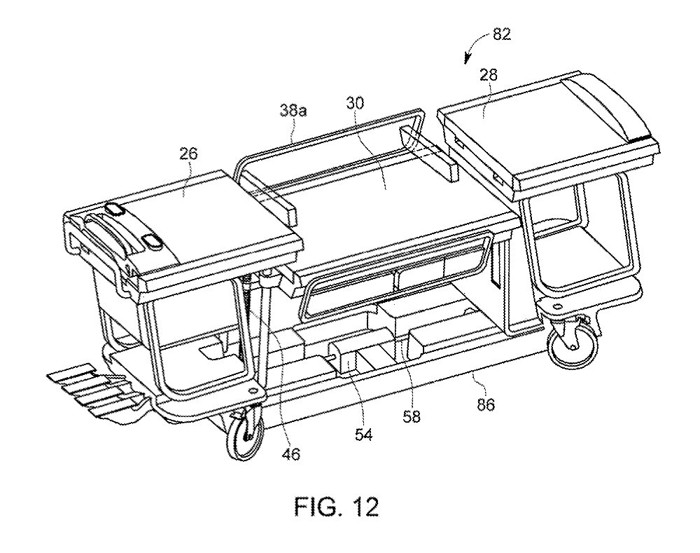

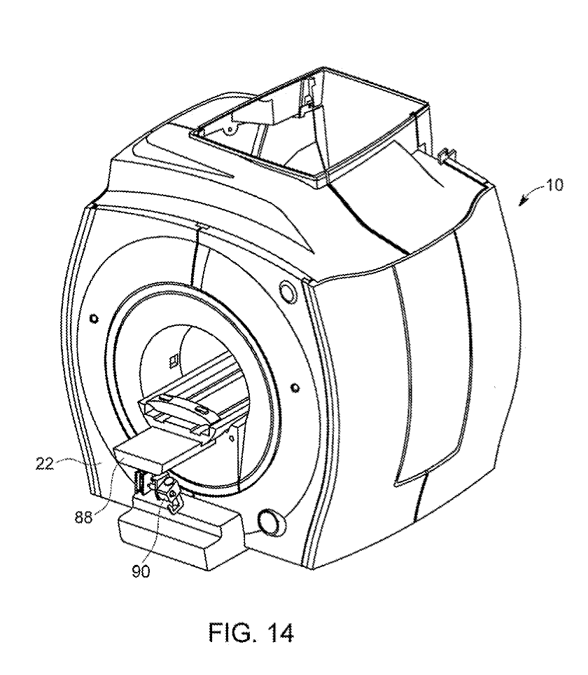

FIGS. 9-15 illustrate a similar, second embodiment, in which the patient support table 82 is mobile and mounted to a series of support wheels 84. The patient support table 82 includes similar first and second sections 26, 28 as well as the movable center section 30. However, in the embodiment shown in the drawing Figures, the mobile patient support table 82 can be moved toward and away from the medical scanning device 10 either with or without the patient supported thereon. The center section 30 is again movable utilizing a similar lift mechanism 44 having a drive motor 54 mounted in the base 86 of the patient support table 82, as best shown in FIG. 12. The drive motor 54 again powers the rotation of a drive shaft 56 which rotates similar lead screws 46 located on opposite ends of the center section 30. However, since the patient support table 82 is mobile and can be located away from the medical scanning device, the patient support table 82 includes a battery backup that can be used to power the drive motor 54 to raise and lower the center section 30.

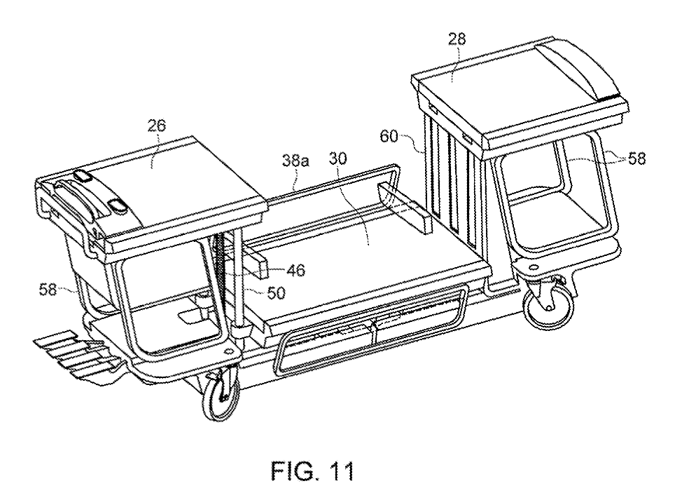

As illustrated in FIG. 11, the first and second sections 26, 28 are again fixed at a first height on a pair of aluminum frames 58. The protective shroud 60 conceals the lead screw 46 and the pair of support rods 50 on either end of the center section 30.

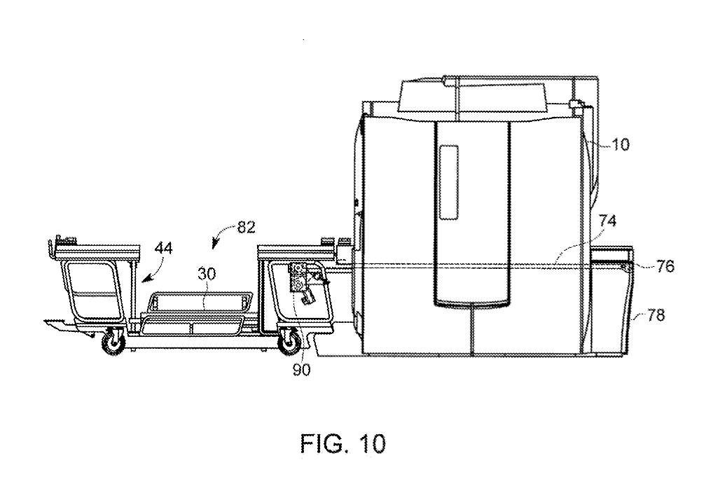

In the embodiment illustrated with a movable patient support table, the medical scanning device 10 includes a bridge extension 88 as best shown in FIG. 15. The bridge extension 88 extends out front the front face 22 and provides a support location for the platform drive mechanism 90. As in the first embodiment, the platform drive mechanism 90 is a dual belt drive, best shown in FIG. 10, that engages a pair of drive belts 74 that extend through the medical scanning device 10 and travel over a pulley 76 located in the rear pedestal 78.

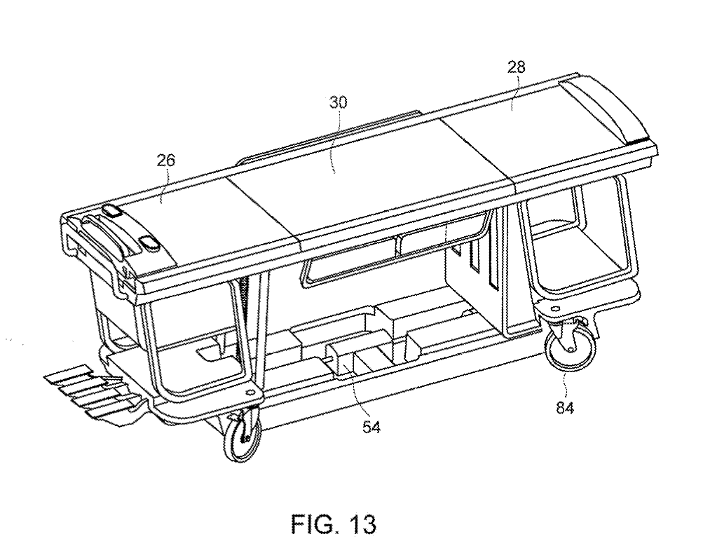

As in the fixed embodiment of the patient support table previously discussed, in the mobile embodiment of the patient support table 82, the center section 30 is movable between the lowered position shown in FIG. 10 and the fully raised position shown in FIG. 13. In the lowered position shown in FIG. 10, the height of the center section is between 350-400 mm where the fully raised position has a height of 965 mm.

In both embodiments, when a patient is initially presented to the patient support table, the center section 30 can be moved to the fully lowered position as shown in FIGS. 3 and 11, respectively. In this position, the patient can sit on the center section 30, which provides easier ingress to the patient support table. Once in this position, an operator can activate the lift mechanism which causes the center section to move from the lower position to the intermediate position shown in FIGS. 5 and 12 and ultimately to the fully raised position shown in FIGS. 6 and 13. Once the patient is in the fully raised position, the patient can lay on the table and prepare for scanning by the medical scanning device 10.

Once the patient is laying on the table, the mechanical and magnetic latching members between the center section 30 and the pair of outer sections 26, 28 allow the entire patient support platform to move as one unit. Movement of the patient support platform is carried out by the platform drive mechanism 72 in the first embodiment and the platform drive mechanism 90 in the second embodiment. The platform drive mechanisms allow the complete support platform to move into and out of the medical scanning device 10 in a known manner.

In the embodiment of the patient supply table shown in the present disclosure, the center section is designed to support at least 750 pounds and to be movable between the lowered position of 350-400 mm to the fully raised position of 950 mm above the floor. In this manner, the patient support table can handle patients of all different sizes, weights and physical abilities. Likewise, if the patient is supported on a stretcher, the center section can be moved to the fully raised position such that the patient can be transferred from the stretcher directly onto the patient support table.

This written description uses examples to disclose the invention, including the best mode, and also to enable any person skilled in the art to make and use the invention. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences front the literal languages of the claims.

I claim:

1. A patient support table to support a patient above a floor, comprising: a patient support platform comprising:
a first section located at a first height;
a second section located at the first height; and
a center section positioned between the first and second sections, wherein the center section is movable between a raised position in which the center section is at the first height and a lowered position in which the center section is at a second height below the first height; and
a lift mechanism mounted to the support table and operable to move the center section between the raised and lowered positions.

2. The patient support table of claim 1 wherein the second height is in the range of 350-400 mm above the floor.

3. The patient support table of claim 1 further comprising at least one arm board mounted to the center section, the arm board is movable between a raised position and a lowered position.

4. The patient support table of claim 3 further comprising a pair of arm boards each mounted to the center section for movement with the center section, wherein each of the pair of arm boards is movable between a raised position and a lowered position.

5. The patient support table of claim 1 wherein the lift mechanism includes a pair of threaded lead screws each coupled to one end of the center section, wherein rotation of the lead screws moves the center section between the raised and lowered positions.

6. The patient support table of claim 5 wherein the lift mechanism includes a drive motor mounted to a base of the support table, wherein the drive motor rotates the lead screws.

7. The patient support table of claim 1 further comprising a platform drive mechanism mounted to the second section and operable to move the patient support platform relative to a base of the support table.

8. The patient support table of claim 1 further comprising a base mounted to a plurality of support wheels such that the patient support table is movable.

9. A patient support table for supporting a patient during a medical scanning procedure by a medical scanning device, comprising:
a patient support platform comprising:
a first section located at a first height;
a second section located at the first height;
a center section positioned between the first and second sections, wherein the center section is movable between a raised position in which the center section is at the first height and a lower position in which the center section is at a second height below the first height;
a lift mechanism operable to move the center section between the raised and lowered positions; and
a platform drive mechanism operable to move the patient support platform, including the first section, second section and the center section into the medical scanning device.

10. The patient support table of claim 9 wherein the first section, the second section and the center section are linked together when located at the first height.

11. The patient support table of claim 10 wherein the platform drive mechanism includes a drive motor coupled to at least one drive belt that extends through the medical scanning device.

12. The patient support table of claim 9 wherein the second height is in the range of 350-400 mm from the floor.

13. The patient support table of claim 9 wherein the platform drive mechanism is mounted to the second support platform.

14. The patient support table of claim 9 further comprising a pair of arm boards mounted to the center section and movable with the center section, wherein each of the pair of arm boards is movable between a raised position and a lowered position.

15. The patient support table of claim 9 wherein the lift mechanism includes a pair of threaded lead screws each coupled to one end of the center section, wherein rotation of the lead screws moves the center section.

16. The patient support table of claim 9 wherein the patient support table is mounted to a base having a plurality of wheels such that the patient support table is mobile.

17. The patient support table of claim 16 further comprising a bridge extension formed on the medical scanning device, wherein the bridge extension engages the second section and wherein the belt drive mechanism is mounted to the bridge extension.

18. A method of moving a patient into and out of a medical scanning device comprising the steps of:
lowering a center section of a patient support platform from a first height;
loading the patient onto the center section while the center section is below the first height;
raising the center section to the first height such that the center section, a first section and a second section of the patient support platform are at substantially the same height; and
operating a platform drive mechanism to move the patient support platform, including the center section, the first section and the second section into the medical scanning device.

19. The method of claim 18 wherein a lift mechanism mounted to the patient support table is operable to move the center section between a raised position and a lowered position.

20. The method of claim 18 wherein the platform drive mechanism is mounted to the patient support table.
